(19) United States
(12) Patent Application Publication
(10) Pub. No.: US 2020/0390698 A1
(43) Pub. Date: Dec. 17, 2020

(54) URETHRAL DEVICES FOR TREATMENT OF PATHOLOGICAL UROLOGICAL CONDITIONS

(71) Applicant: INURA MEDICAL AG

(72) Inventors: Otto von Stetten, Aachen (DE); Jean-Jaques Becciolini, Geroldwil (CH); Terry Nida, St. Marys, GA (US)

(73) Assignee: INURA MEDICAL AG, Luzern (CH)

(21) Appl. No.: 16/074,242

(22) PCT Filed: Feb. 1, 2017
(86) PCT No.: PCT/EP2017/052131

§ 371 Date: Jul. 31, 2018

(30) Foreign Application Priority Data
Feb. 2, 2016 (GB) 1601855.8

(51) Int. Cl. A61K 9/02; A61K 9/00; A61K 31/277; A61K 47/44; A61K 47/02

(52) U.S. Cl. CPC A61K 9/025; A61K 9/0034; A61K 47/02; A61K 47/44; A61K 31/277

(57) ABSTRACT

A newly identified problem with conventional treatments of overactive bladder and nocturia is that these diseases are not simply due to dysfunctional bladder detrusor smooth muscle, but rather is a complex disorder involving interplay between sensory and motor nerves. Thus, although the use of anti-muscarinic agents alone can be useful in some women, we discovered that treatment therapy using agents that have mixed-activity anti-cholinergic activity can be especially useful. Further, because absorption of a drug across cell layers is affected by the lipid solubility of the drug, this invention also optionally includes adjusting the pH of the suppository to affect the relative concentrations of charged (polar) and uncharged (non-polar) forms of the drug.

### BACKGROUND

This invention is directed to buffered urethral suppositories to treat the symptoms of acute urinary urgency and frequency associated with the diseases of nocturia and overactive bladder (OAB), including methods for their use. In particular, this invention relates to suppositories incorporating cholinergic and/or mixed anti-cholinergic agents and uses thereof to treat symptoms of nocturia and overactive bladder.

Overactive bladder is defined as urgency with or without urge incontinence, usually with frequency and nocturia. This condition is characterized by involuntary bladder muscle contractions during the time the bladder is filling with urine. Such muscle contractions may be spontaneous or provoked with patients unable to suppress them, resulting in involuntary expression of urine.

Overactive bladder is a common malady afflicting more than 100 million women worldwide. Importantly, the disease worsens as women age resulting with one in four women aged 65 or older presenting with the disease.

While not life-threatening for the afflicted woman, her quality of life may be negatively impacted due to the inconvenience, potential embarrassment, and disrupted sleep patterns associated with overactive bladder. Frequently these women are afraid to leave their home, or are unable to participate in a lengthy meeting, dinner, social and/or travel event. Unfortunately, many of these women fail to seek treatment, as they perceive their symptoms to be a normal part of the aging process. Such a perception is incorrect as an overactive bladder is not normal, is treatable, and treatment can significantly ameliorate symptoms, thus improving the woman's quality of life.

Certain aspects of the present invention include a urethral suppository comprising:
1) A carrier base material;
2) A therapeutically effective amount of an agent selected from the group consisting of an anti-cholinergic and/or a mixed-activity anti-cholinergic agent; and optionally
3) A buffering agent in a sufficient quantity to ensure that an effective amount of the therapeutic agent is in an uncharged state for dissolution in the urethra; wherein the suppository is formed into a solid structure configured for insertion into the urethra of a female patient.

Additional aspects of this invention include use of urethral suppositories to treat symptoms of nocturia and overactive bladder.

### DETAILED DESCRIPTION

The distal portion of the urinary tract includes the bladder and urethra. The bladder stores urine produced by the kidneys until voiding becomes necessary. The bladder is under control of the autonomic nervous system, including sensory (afferent) nerves and parasympathetic (efferent) nerves. The urethra is a hollow tubular tissue structure that connects to and provides an external outlet (the meatus) for urine from the bladder.

In women the urethra is approximately 4 to 5 centimeters in length. Near the proximal end of the urethra, where it attaches to the bladder, is the internal sphincter, which is under involuntary reflex control and opens in response to pressure from the bladder when abdominal muscles bear down on the bladder. The external sphincter is located near the distal end of the urethra in close proximity to the meatus and is under voluntary control thereby allowing urine evacuation under voluntary control.

Urethral sphincter control is involved in one of the symptoms of nocturia and overactive bladder, incontinence. One type of incontinence is called urge incontinence, which occurs when muscle spasms of the bladder result in sensory urgency and leakage from the internal urethral sphincter that has opened in response to increased bladder pressure. Of note, the internal urethral sphincter muscle is unusual in that in its normal state the smooth muscle is contracted and to open the sphincter the smooth muscle needs to relax. Since the urethra controls incontinence at two of its sphincters and incontinence is often associated with overactive bladder, the urethra is an important part of overactive bladder.

Limited systemic perfusion of the urethra makes oral or systemic delivery of drugs poor routes of administration to treat nocturia, overactive bladder and associated urethral disorders, as such drugs would have difficulty gaining access to the urethra and may have to be administered at such a high dose that systemic or other side-effects make the drug less tolerable. However, the alternative, local delivery of drug to the urethra poses its own special problem due to the periodic flushing of urine through the urethra, which would wash away any unabsorbed drug on the epithelium of the urethra. Furthermore, urine can vary significantly in pH from 4.5 to 8 which would dramatically change the absorbability of charged molecules whose charge is pH dependent resulting in unpredictable drug absorption from the urethra. Consequently, there is a need to treat overactive bladder via the urethra locally but solve the problem of the drug being washed away prematurely before being absorbed by urine flow and to allow rapid absorption in spite of the tremendous variability in the pH of the local environment due to remnant urine on the internal surface of the urethra.

Although overactive bladder is considered a disease of the bladder, researchers have described a urethral component to the disease. This is not surprising as many neural and systemic networks are shared by the bladder and urethra. Unlike the bladder, the urethra is a collapsed tube in its resting stage and opens to allow urine to pass out of the bladder. Therefore, any liquid or gel material placed in the urethra due to capillary pressure would be pushed out of the urethra into the bladder or out of the urethra via the meatus due to contraction of the external sphincter. To treat the urethra, a medication can be incorporated into a structure that is retained in the urethra for a period of minutes to hours. Embodiments of a drug delivery system include a suppository base as a means to expose the urethra to medication for a period of time from minutes to hours. The formulation of the delivery system including the type of base materials used as a delivery vehicle, the concentration of drug, and the ratio of drug to buffering agent can be chosen so as to produce an efficient mechanism for delivering the therapeutic agent. Size can be an important aspect for the performance of a urethral suppository and for patient tolerance of the suppository, and thus is a consideration in any suppository formulation. The female urethra is approximately 4-5 cm in length. Most patients can tolerate an object placed in their urethras up to about 19 French (6.3 mm) in size without major discomfort. The maximum size of a urethral suppository that comfortably fits in the female urethra is about 2.5 cm in length and about 0.65 cm in diameter or transverse dimension.

In the more developed healthcare systems around the world, oral anti-cholinergic drugs are the mainstay of treatment for this disease state. However, we have discovered this standard of care is deficient. Patient compliance studies report that more than half of patients taking an oral OAB drug stop taking it within six months of initiation of therapy. Such studies also report that only 10 to 20 percent of patients remain on an oral OAB medicine 12 months after initiating treatment. About a third to one-half of those who discontinue their drug therapy do so due to side effects they cannot tolerate or do not find the minimal benefit they receive to outweigh the negative side effects of their prescribed drug.

Manufacturers of these oral overactive bladder therapies have expended significant research energy and money in their efforts to reduce side effects to increase patients' adherence to treatment. However, most physicians, health experts and healthcare providers do not believe the marginal benefits of these oral agents outweigh the significant side effects experienced by patients prescribed such drugs. Consequently there is a significant need for more effective treatments for overactive bladder.

Based on our discovery that overactive bladder can result from an interaction between motor and sensory nerves this invention provides new therapeutic alternatives that can supplement or supplant conventional therapies.

In certain embodiments, the present invention provides urethral suppositories and methods for their use in treatment of symptom(s) of overactive bladder. Such symptoms can include sensations of urgency, frequency, nocturia, pain and/or incontinence. For example, such sensations can be a result of bladder or urethral irritation due to urethritis or an indwelling catheter. Components of a suppository of this invention include a therapeutic agent that is an anti-cholinergic or mixed-activity anti-cholinergic agent. The therapeutic agent is present in a sufficient quantity to provide a therapeutic effect. The components of the suppository also include a buffering agent; the buffering agent is present in a sufficient quantity to ensure that a therapeutically effective amount of the therapeutic agent is in an uncharged state for dissolution in the urethra. This uncharged state of the compound would represent at a minimum of 10% of the total drug up to 100% in its unchanged state. For the 10% minimum, this would require an amount of buffer to keep the compound within 1 pH unit of the compound's pKa. However, if the compound is fully uncharged, at pH units further away from the pKa, then the pH of the buffer is present to ensure tissue tolerability, which for the urothelium is pH range 4.5-9.0, somewhat outside the pH range of human urine. The therapeutic agent and the buffering agent are in a carrier base material.

Base Suppository Material

In some embodiments, a carrier base material is an excipient base composed of a polymer, hydrophobic, wax-like or glycerin base that is meltable or dissolvable at body temperature. The carrier base material can have a melting point such that the suppository is substantially melted at body temperature. In some embodiments, the base material or base materials have a melting point of from about 36° C. to about 38° C. The melting time of embodiments of the carrier base material or combination of carrier base materials can be adjusted by the addition of paraffin to the liquid mixture to achieve a melting time from about 5 minutes to about 15 minutes. Alternatively, the carrier base material can be a water-soluble carrier base.

In some embodiments, the carrier base material is selected from the group consisting of paraffin, hexanone, theobroma oil, modified theobroma oil products, gelatins, glycerinated gelatins, polyethylene glycols (PEGs), glycerols, hydrogenated vegetable oils, cocoa butter, celluloses, polyvinyl alcohol, polyvinylpyrrolidone, polyacrylamide, polyphosphourethanes, polyoxyl stearate, ethylene oxide polymers, and fatty acid bases and the like. These materials can be used individually or in combination.

The carrier base material can further include additional components such as preservatives. Suitable preservatives include, but are not limited to methylparaben, ethylparaben, propylparaben, butylparaben, chlorphenesin, chlorobutanol, sorbic acid, thimerosal, and other preservatives commonly used in the art for pharmaceutical compositions, including suppositories. Typically the preservative is methylparaben or propylparaben.

The carrier base material can further include an alkali as a pH adjusting agent to adjust the buffer pH to an appropriate value. The alkali can be, but is not limited to, sodium carbonate, sodium hydroxide, magnesium oxide, or another alkali. The alkali is to be distinguished from the buffering agent.

The composition of some embodiments, and particularly some of the buffering agents and/or therapeutic agents, can include pharmaceutically acceptable salts. Pharmaceutically acceptable salts include, but are not limited to, acetate, benzenesulfonate, besylate, benzoate, bicarbonate, bitartrate, bromide, calcium edetate, carnsylate, carbonate, citrate, edetate, edisylate, estolate, esulate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mutate, napsylate, nitrate, pamoarte (embonate), pantothenate, phosphate/diphosphate, polygalacturonate, salicylate, stearate, subacetate, succinate, sulfate, tannate, tartrate, or teoclate. Other pharmaceutically acceptable salts can be found in, for example, Remington, The Science and Practice of Pharmacy (20^{th} ed, Lippincott, Williams & Wilkins (2000), which is incorporated herein in its entirety by reference.

Silica can also be added as a suspending agent to prevent the active ingredients within the suppository from aggregating. Silica can comprise from about 0.1 percent to about 5 percent by weight of a suppository according to the present invention. Other suspending agents can alternatively be used and are well known to those skillful in the art.

Additionally, still other ingredients can be included in a suppository according to the present invention. Such ingredients can include, for example, a coloring agent, an antioxidant, a chelating agent, a preservative and other ingredients typically used in pharmaceutical formulations, including in suppositories.

Although anti-cholinergic agents, including anti-muscarinic agents, can be effective in relieving some symptoms involving undesirable contractions of bladder musculature, in many patients however, such pure anti-muscarinic smooth muscle relaxants provide incomplete or ineffective treatment. Thus, in other aspects, sensory pathways can play important roles in maintaining the pathophysiological overactive state. Thus, by intervening in sensory pathways, this invention provides alternative therapeutic approaches. Anti-cholinergic agents that may be used in the scope of this invention include darifenacin, fesoterodine, flavoxate, mirabegron, oxybutynin, probantheline, solifenacin, tolterodine, and/or trospium etc. and mixtures thereof, as well as mixtures with mixed activity anti-cholinergic agents.

Roles of Sensory Nerves and Inflammation in Overactive Bladder

In the urinary tract, sensory nerves are considered to be part of the autonomic nervous system. There are several types of sensory nerves that help regulate the bladder. For example, sensory c-fibers (small, unmyelinated fibers) contain neuropeptides, including the neurokinins substance P (SP), neurokinin A and neurokinin B, vasoactive intestinal peptide (VIP), and others. Neurokinins act via neurokinin receptors, in which, neurokinin 1 receptors are most sensitive to substance P, neurokinin 2 receptors are most sensitive to neurokinin A, and neurokinin 3 receptors are most sensitive to neurokinin B. Significantly, when such sensory nerves are activated, SP can be released into the medium surrounding the nerve and adjacent structures. When released, SP can cause sensations of pain, burning, and other unpleasant sensations. We believe that such sensations may be at least in part responsible for the discomfort of overactive bladder.

Sensory Nerve Therapies

Therefore, other aspects of this invention include suppositories and methods using agents that interfere with aspects of the sensory nervous system and/or inflammatory cascades. In some embodiments, a mixed-activity anti-cholinergic agent can reduce the sensitivity of sensory nerves. Although not bound by any particular mechanism of action certain of such agents can interfere with voltage-dependent sodium channels. By interfering with sensory nerve activation and/or transmission, certain mixed activity anti-cholinergic agents can reduce sensor and/or motor nerve activity.

Mixed-Activity Anti-Cholinergic Agents

In some embodiments, a therapeutic agent can have multiple drug activities including its anti-cholinergic activities and hence is termed a mixed-activity anti-cholinergic drug. For example, many tricyclic antidepressants exhibit significant anti-cholinergic activity at their therapeutic drug levels used to treat depression. Typically, the mixed-activity anti-cholinergic drug selected from the group consisting of: amineptine, amitriptyline (pKa 9.4), amiltriptylinoxide, amoxapine, butriptyline, cidoxepin, clocapramine, clomacran, clomipramine (pKa 9.5), daledalin, demexiptiline, desmethylamitriptyline, desipramine, dibenzepin, dimetacrine, dotheipin, doxepin (pKa 8.0), fluacizine, N-methyl-doxepin, imipramine (pKa 9.5), iprindole, lofepramine, maprotiline, melitracin, metapramine, mirtazepne, nitroxazepine, norclolipramine, nortriptyline (pKa 9.7), noxiptyline, opipramol, perlapine, pizotyline, propizepine, protriptyline (pKa 8.2), quinupramine, reboxetine, tianeptine, trimipramine (pKa 9.5), and pharmaceutically acceptable derivatives and bioisosteres thereof. In some embodiments, a mixed-activity anti-cholinergic drug is doxepin. It should be appreciated that by including such mixed-activity anti-cholinergic agents in a local delivery system such as a urethral suppository, undesirable central effects can be minimized or even eliminated.

The therapeutic effect of all of these therapeutic agents can increase when mixed with suitable buffers. However, by mixing buffering agents at different concentrations with these therapeutic agents, different therapeutic effects over varying periods of time can be achieved. It can be useful for some embodiments to have a therapeutic agent that is configured to inhibit the conduction or initiation of nerve impulses. Typically, the quantity of therapeutic agent incorporated in a suppository according to the present invention is sufficient to ameliorate a urethral disorder. Some embodiments of the urethral suppository can include about 1 mg to about 100 mg of therapeutic agent; typically, the urethral suppository includes about 30 mg to about 60 mg of therapeutic agent.

Buffering Agents

In other aspects of this invention, a buffer may be included to control absorption of an active agent into the urethra. When used, typically, the buffering agent is present in a quantity such that the buffering agent buffers the therapeutic agent in the dissolved/melted suppository at a pH that ensures a sufficient portion of the therapeutic agent is present in the dissolved/melted suppository in an uncharged state so that the therapeutic agent can cross cell membranes of cells surrounding the urethra. Typically, the buffering agent maintains the pH of the dissolved/melted therapeutic agent so that at least about 10% up to 100% of the therapeutic agent is present in its uncharged state. Typically, this would be within 1 pH unit of the drug's pKa. However, the therapeutic agent can be buffered at a pH further away from its pKa as long as it is present in its uncharged state, which would allow rapid permeation into urethral epithelium and subtending tissues. Consequently, the pH of the buffer would depend on the therapeutic agent, its pKa and its amount provided in the suppository. This pH range would be allowed to vary significantly since the urothelium can tolerate pH ranges from 4.5 to over 8.0. The pH range of the buffer would range from about 4.5 to about 10.0.

In certain embodiments of this invention, the buffering agent is selected from the group consisting of sodium bicarbonate buffer, calcium bicarbonate buffer, tris (hydroxymethyl) aminomethane (Tris or THAM), MOPS (3-(N-morpholino) propanesulfonic acid) buffer, HEPES (N-(2-hydroxyethyl) piperazine-N1-(2-ethanesulfonic acid) buffer, ACES (2-[(2-amino-2-oxoethyl)amino)ethanoesulfonic acid) buffer, ADA (N-(2acetamido)2-iminodiacetic acid) buffer, AMPSO (3-[(1,1-dimethul-2-hydorxyethyl)amino]-2-propanesulfonic acid) buffer, BES (N,N-bis(2-hydroxyethyl)-2aminoethanesulfonic acid) buffer, Bicine (N,N-bis(2-hydroxyethylglycine) buffer, Bis-Tris (bis-(2hydroxyethyl)imino-tris(hydroxymethyl) methane buffer, CAPS (3-(cyclohexylamino)-1-propanesulfonic acid) buffer, CAPSO (3-(cyclohexylamino)-hydroxy-1-propanesulfonic acid) buffer, CHES (2-(N-cyclohexylamino) ethanesulfonic acid) buffer, DIPSO (3-[N,N-bis(2-hydroxyethyl) amino)-2-hydroxy-propanesulfonic acid) buffer, HEPPS (N-(2-hydroxyethylpiperazine)-N1-(3-propanesulfonic acid) buffer, HEPPSO (N-(2-hydroxyethyl) piperazine-N1-(2-hydroxypropanesulfonic acid) buffer, MES (2-(N-morpholino ethanesulfonic acid) buffer, triethanolamine buffer, imidazole buffer, glycine buffer, ethanolamine buffer, phosphate buffer, MOPSO (3-(N-morpholino)-2-hydroxypropanesulfonic acid) buffer, PIPES (piperazine-N,N1-bis(2-ethanesulfonic acid) buffer, TAPS (N-tris[hydroxymethyl)methyl-3aminopropane sulfonic acid) buffer, TAPSO (3-[N-tris(hydroxymethyl)methylamino]-2-hydroxy-propanesulfonic acid) buffer, TES (N-tris(hydroxymethyl)methyl-2-aminoethanesulfanic acid) buffer, tricine (N-tris(hydroxymethyl)methylglycine buffer), 2-amino-2-methyl-1,3-propanediol buffer, and 2-amino-2-methyl-1-propanol buffer or another buffer that can buffer the composition to be administered at the appropriate pH value. In certain embodiments buffering agents include sodium bicarbonate buffer and tris(hydroxymethyl)aminomethane buffer.

The buffer to be selected, and the concentration of the buffer to be used, can be chosen by one of ordinary skill in the art to buffer the composition to be administered at a pH value that is close to the isoelectric point of the local therapeutic drug. For doxepin the pH value is 8.0. Typically, the pH achieved by the use of the buffer is between about 4.5 and about 10.0 but again is dependent upon the pKa of the drug to be delivered and the pH tolerability of the urethra. More typically, the pH achieved by the use of the buffer is between about 7.0 and about 9.5. Typically, when bicarbonate buffer is used, it is sodium bicarbonate buffer; however, other counter-ions can be used as described above.

In cases in which the pKa of the therapeutic agent is near neutral, pH 7.0, the buffer may be unnecessary; however, even for therapeutic agents in which the pKa of the drug is near neutral, the use of a buffer is typically preferred to maintain a suitable pH and prevent undesirable fluctuations in pH, which could otherwise lead to undesirable fluctuations in drug delivery.

Buffering agents that can be used for embodiments of the suppository can include buffering agents that are present in a quantity such that the buffering agent or agents buffers the suppository at a pH that ensures that a sufficient portion of a therapeutic agent that is present in the suppository is present in an uncharged state so that the therapeutic agent can cross cell membranes of cells surrounding the urethra. Typically, the buffering agent can maintain a pH of the urethral suppository in a range of from about 7 to about 12. More typically, the buffering agent can maintain a pH of the urethral suppository in a range of from about 7 to about 9.

Some embodiments of the urethral suppository can include from about 0.5 mg to about 100 mg of buffering agent. Typically, the urethral suppository includes from about 1 mg to about 20 mg of buffering agent. Some embodiments of the urethral suppository can contain a quantity of buffering agent that comprises from about 1 percent to about 30 percent by weight of the overall weight of the suppository. Typically, the urethral suppository contains a quantity of buffering agent that comprises from about 5 percent to about 10 percent by weight of the overall weight of the suppository.

Manufacture of Urethral Suppositories

Typically, the suppository is from about 10 mg to about 2000 mg in weight; more typically, from about 400 mg to about 600 mg in weight. The suppository typically comprises from about 1 mg to about 100 mg of therapeutic agent. More typically, the suppository comprises from about 30 mg to about 60 mg of therapeutic agent. Typically, the suppository comprises from about 0.5 mg to about 100 mg of buffering agent; more typically, the suppository comprises from about 1 mg to about 20 mg of buffering agent. Typically, the suppository is in a configuration selected from the group consisting of a baseball bat, a cylinder, a cone, a rocket and an ellipsoid. In one alternative, the suppository is an elongated structure with a transverse dimension of from about 1 mm to about 10 mm; typically, in this alternative, the transverse dimension is from about 3 mm to about 6 mm. In another alternative, the suppository is an elongated structure with a length of from about 5 mm to about 50 mm; typically, in this alternative, the length is from about 15 mm to about 35 mm. Typically, the suppository comprises a quantity of buffering agent that comprises from about 1 percent to about 30 percent by weight of the overall weight of the suppository.

A suppository can further comprise a quantity of a suspending agent sufficient to prevent active ingredients within the suppository from aggregating. The suspending agent can be silica.

Another aspect of the present invention is a method for manufacturing a urethral suppository comprising the steps of: (1) Combining a therapeutic agent selected from the group consisting of a mixed-activity anti-cholinergic agent and a buffering agent in a liquid carrier base material until the therapeutic agent and the buffering agent have dissolved or been suspended in the liquid carrier base material; and (2) forming the liquid carrier base material, therapeutic agent, and buffering agent mixture into a suppository that is configured to be deployed within the urethra of a patient.

Typically, the step of forming the mixture into a suppository results in a finished suppository having a weight of from about 10 mg to about 1000 mg.

Some embodiments of a method for manufacturing a urethral suppository include combining a therapeutic agent selected from the group consisting of a mixed-activity anti-cholinergic agent and a buffering agent in a liquid carrier base material until the therapeutic agent and the buffering agent have dissolved or been suspended in the liquid carrier base material. The mixture of the liquid carrier base material, the therapeutic agent, and the buffering agent is then formed into a suppository that is configured to be deployed within a patient's urethra. Typically, the step of forming the mixture into a suppository results in a finished suppository having a weight of from about 10 mg to about 1000 mg. Typically, the quantity of buffering agent combined with the therapeutic agent in the liquid base material is sufficient to produce a pH of from about 4.5 to about 10.0 in the finished suppository.

Urethral suppositories, according to certain embodiments of the present invention, can be formed by mixing or combining components into a mixture and forming the mixture into a solid suppository. For example, such methods can include combining one or more therapeutic agents and a buffering agent in a liquid carrier base material. The combined components can be stirred, heated, or both until the components have been dissolved or suspended in the liquid carrier base material. For example, the carrier base material can be warmed in a bath having a temperature of from about 35° C. to about 45° C. After the therapeutic agent has dissolved or been otherwise suspended in solution, the mixture can be heated or have the temperature raised to a temperature of from about 60° C. to about 80° C. Thereafter, the mixture can be titrated with a buffering agent, such as sodium bicarbonate, to a pH of from about 7.0-8.0 while gently stirring the mixture. In some embodiments, the mixture may lose its grainy appearance and become clear as all components are all dissolved in the carrier base material. Once the components have been suitably mixed, the liquid carrier base material, therapeutic agent, and buffering agent mixture can be formed into a suppository or other type of solid depot that is configured to be deployed within a patient's urethra or other body lumen.

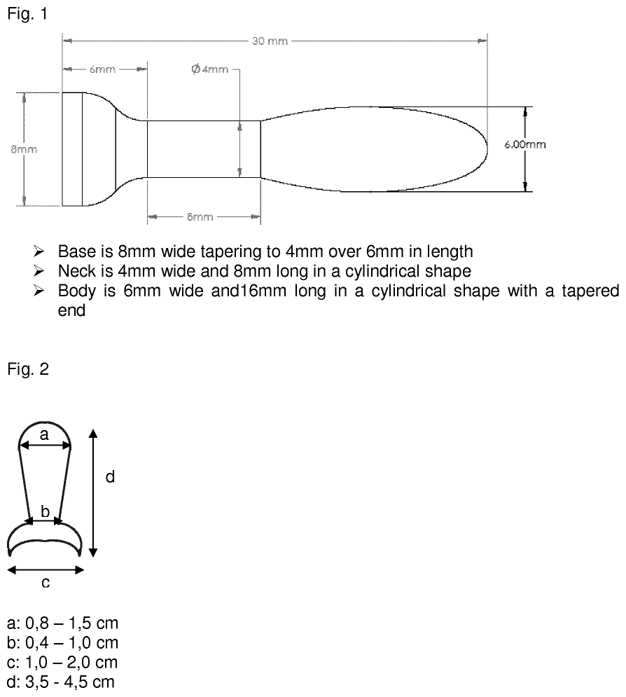

In some embodiments, a suppository has an elongated cylindrical configuration with rounded or spherically shaped ends; however, other configurations such as conical or ellipsoidal can alternatively be used. As discussed above, the ellipsoidal can alternatively be used. As discussed above, the size of the suppository is a significant variable in some applications. In particular, for urethral suppositories, it can be useful to have as large a suppository as possible while maintaining an acceptable level of patient discomfort. Some embodiments of a urethral suppository can have a length of about 5 mm to about 50 mm; typically, the length is from about 15 mm to about 35 mm. Such embodiments can have a transverse dimension of from about 1 mm to about 10 mm; typically, the transverse dimension is from about 3 mm to about 6 mm. The overall weight of some embodiments of a suppository can be from about 10 mg to about 1000 mg; more typically, the overall weight is from about 50 mg to about 750 mg; preferably, the overall weight is from about 400 mg to about 600 mg. The overall weight can be chosen depending on the appropriate size and shape of the suppository. Examples of different shapes are given in FIG. 1 and FIG. 2.

Typically, the quantity of buffering agent combined with the therapeutic agent in the liquid base material is sufficient to produce a pH of from about 4.5 to about 9.5 in the finished suppository. The suppositories can also be stored at lowered temperatures, such as from about 0° C. to about 10° C. prior to use.

The following formulation examples illustrate the invention without limiting the scope:

| Oxybutynin | 4 mg | 4 mg | 4 mg | 4 mg |
|---|---|---|---|---|
| Na Bicarb | 10 mg | 10 mg | 10 mg | 10 mg |
| Base | 450 mg | 450 mg | 450 mg | 450 mg |
| MBK:Paraffin | 90:10 | 85:15 | 80:20 | 70:30 |

| | |
|---|---|
| Oxybutynin | 2 mg |
| Na Bicarb | 5 mg |
| Base | 450 mg |
| MBK:Paraffin | 85:15 |

Na Bicarb is Sodium Bicarbonate buffer
MBK base is comprised of Theobroma oil, glyceryl monostearate in triglycerides, Hexanone
Base is comprised of MBK base and paraffin

The ingredients were mixed and melted, under stirring poured into the appropriate forms and let cool to room temperature.

Methods of Use

In methods according to the present invention, the exact formulation and dosage used in suppositories according to the present invention can be selected by the individual physician with respect to the patient's condition. (See e.g. Fingl et al., in The Pharmacological Basis of Therapeutics, 1975, Ch. 1 p. 1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administered dose in the management of the disorder of interest will vary with the severity of the condition to be treated, the general condition of the urinary tract, including the bladder and urethra, and the existence of other conditions affecting the urinary tract, such as infections, inflammation, or allergic reactions. The severity of the condition may, for example, be evaluated, in part, by standard prognostic evaluation methods. Further, the dose, and perhaps the dose frequency, will also vary according to the age, body weight, and response of the individual patient. A program comparable to that discussed above may be used in veterinary medicine.

Another aspect of the present invention is a method of treating at least a portion of a patient's urinary tract comprising the steps of:
(1) providing a urethral suppository according to the present invention as described above;
(2) deploying the urethral suppository within the patient's urethra; and
(3) allowing the suppository to at least partially disintegrate and release the mixed-activity anti-cholinergic therapeutic agent and the buffering agent to treat at least a portion of the urinary tract of the patient.

Disintegration of the suppository can comprise melting of the carrier base material of the suppository. Alternatively, disintegration of the suppository can comprise dissolving the carrier base material of the suppository.

When the therapeutic agent is mixed-activity anti-cholinergic drug, as described above, treating at least a portion of the urethra can comprise treatment of overactive bladder or one of its symptoms as previously described.

Embodiments of urethral suppositories include formulations including a therapeutic agent selected from the group consisting of a mixed-activity anti-cholinergic agent, which is included in the suppository mixture. Typically, the therapeutic agent is present in a quantity sufficient to prevent or ameliorate at least one symptom associated with a urinary tract disorder. The symptom that is prevented or ameliorated can be one or more of urgency, frequency, nocturia, or incontinence. Typically, the urinary tract disorder with which at least one symptom is associated is overactive bladder, but suppositories according to the present invention can also be formulated for the treatment of other urinary tract disorders, such as, but not limited to, interstitial cystitis, painful bladder, detrusor instability, or other urinary tract disorders associated with one or more of these symptoms.

As described above, lipid molecules are a primary component of cell membranes and are known to be hydrophobic. In the bladder and urethra, the lipid membranes of the urothelial cells are shielded from direct contact with urine by the presence of the GAG (glycosaminoglycan) barrier. GAG molecules readily attract water molecules creating a hydrated mucous layer that is a primary component of the layer that lines the urethra and the bladder luminal wall. The water molecules in the hydrated mucous layer have a slight negative charge that repels other negatively charged ionic species. However, uncharged therapeutic agent molecules, such as doxepin, as described above, are nonpolar when at a pH above its pKa and as such are more lipophilic than hydrophilic; such uncharged therapeutic agent molecules can still readily pass through the hydrated mucous layer. Accordingly, they easily and efficiently pass through the mucous layer and cell membranes of the bladder and urethral tissue cells.

Once the urethral suppository has been formed or otherwise manufactured, it can be deployed within the body of a patient in order to treat overactive bladder, another disease or condition affecting the urinary tract, or a symptom associated with overactive bladder or another disease or condition affecting the urinary tract.

Another aspect of the present invention is a method of treating at least a portion of the urinary tract of a patient comprising the steps of:
(1) providing a urethral suppository according to the present invention as described above;
(2) deploying the urethral suppository within the patient's urethra;
(3) allowing the suppository to at least partially disintegrate and release the therapeutic agent and the buffering agent to treat at least a portion of the urinary tract of the patient.

In certain alternatives, disintegration of the suppository comprises melting of the carrier base material of the suppository. In another alternative, disintegration of the suppository comprises dissolving of the carrier base material of the suppository.

Once the urethral suppository has been manufactured, it can be deployed within the body of a patient in order to treat a variety of symptoms and conditions as described herein. Suppositories and methods of their use can also be applied to veterinary medicine to treat similar indications in animals. For example, they could be used to treat a socially or economically important animal such as a horse, a cow, a sheep, a goat, a donkey, a mule, a dog, a cat, a pig, or another socially or economically important animal.

In some embodiments a suppository of this invention can be inserted using a catheter. A delivery catheter can be an elongated tubular member having a length of about 10 cm to about 100 cm, an outer transverse dimension or diameter of about 19 Fr. And an inner lumen having an inner transverse dimension or diameter of up to about 10 mm. The delivery catheter can have any suitable construction, including that of an extruded polymer tube that can optionally be reinforced with braided material or the like. Materials such as polyethylene, polyurethane, nylon, or the like can be used. Once the suppository has been advanced to the distal portion of the delivery catheter and ejected from a distal end of the delivery catheter, the delivery catheter can be withdrawn from the urethra of the patient.

In certain embodiments, a suppository has a shape similar to that of a baseball bat. Such a suppository has a first end and a second end; the suppository is inserted into the urethra of a patient such that the first end enters the urethra first. The first end terminates in a generally conical tip. The second end terminates in a pair of protrusions. The shape of the suppository is such that it tapers gradually in diameter from the first end to a point that is typically located about one-fifth to one-eighth of the total length of the suppository from the second end. The suppository then increases in diameter from that point to the second end. Typically, in this embodiment, the suppository has a total length of 2.5 cm (1 inch). However, suppositories according to the present invention can be manufactured in other lengths with this shape. This shape is exemplary and can be modified.

Efficacy of Therapy

Efficacy of therapy using embodiments of this invention can be monitored by patient history, clinical findings, laboratory findings, subjective impressions of the patient and/or other methods. In the event that therapy is incomplete under one circumstance, dosages and frequencies of administration can be varied. The exact dosage delivered to the patient can depend on the subject to be treated, the age of the subject to be treated, the body weight of the subject to be treated, the nature of the disease or condition for which the suppository is administered, the severity and course of the disease or condition of the subject to whom the suppository is administered, the response of the subject, and pharmacokinetic considerations such as liver and kidney function that affect the metabolism of any administered therapeutic agent. The optimal concentration and dosage of the therapeutic agent to be delivered can also depend on the specific therapeutic agent used, the buffering agent used, the carrier base material used, and any optional ingredients used in the suppository. These factors can be determined by those of skill in the medical and pharmaceutical arts in view of the disclosure of the present application. Generally, a therapeutically effective dose is desired. A therapeutically effective dose refers to that amount of the therapeutic agent that results in a degree of amelioration of symptoms prior to treatment.

As used herein, the term “treats” or equivalent terminology refers to any detectable improvement, whether subjective or objective, in the urinary tract disorder of the subject to whom the composition is administered. For example, the term “ameliorate” can refer to an improvement as determined by the PORTS scale, PUF scale, or any component of those scales; reduction of pain; reduction of urinary frequency; reduction of urinary urgency; reduction of nocturia; reduction of incontinence; or improvement in more than one of these parameters.

The term “ameliorate” does not state or imply a cure for the underlying condition such as overactive bladder. The dosage forms containing effective amounts are within the bounds of routine experimentation, and therefore, are well within the scope of the embodiments disclosed herein. In general, however, a suitable dose of a buffered therapeutic agent for topical delivery via the suppository route can be in the range of from about 0.1 to about 10 mg/kg of body weight per day, typically in the range of from about 0.4 to about 2 mg/kg of body weight per day.

Advantages

Suppositories according to the present invention provide an effective and simple means for treatment of one or more symptoms of overactive bladder. They are well tolerated and can be utilized by most patients. When suppositories according to the present invention are administered, they provide an efficient means of delivering a therapeutic drug agent to tissues of the urinary tract without risking the possibility of excessive systemic doses of the therapeutic drug agent. Because a suppository of the present invention includes a mixed-activity anti-cholinergic agent having both end-organ anti-cholinergic and sensory nerve effects, the dual effects can act synergistically to produce effective therapy without undesirable side effects. Such suppositories can therefore be useful in patients in which pure anti-muscarinic agents are ineffective.

Suppositories according to the present invention possess industrial applicability as manufactured articles that can be used to treat a variety of diseases and conditions of the urinary tract, including, but not limited to, overactive bladder.

The inventions illustratively described herein can suitably be practiced in the absence of any element or elements, limitation or limitations, not specifically disclosed herein. Thus, for example, the terms “comprising,” “including”, “containing”, etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and sub-generic groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of a number of different alternatives, it can be appreciated that the scope of the invention includes any individual member or sub-group of members of the alternatives.

It is also understood that the above description is intended to be illustrative and not restrictive. Many embodiments will be apparent to those experienced in the art upon reviewing the above description. The disclosures of all articles and references, including patient publications, are incorporated herein by reference.

1. A suppository to treat urological disorders in the human and veterinary field comprising:
a) a therapeutic agent selected from anti-cholinergic agents and/or mixed-activity anti-cholinergic agents; and
b) a fatty base and/or a water soluble base.

2. A suppository according to claim 1, wherein the suppository is adapted to be placed in an urethra.

3. A suppository according to claim 1, additionally comprising an osmolar component.

4. A suppository according to claim 1, wherein the suppository melts or dissolves.

5. A suppository according to claim 1, wherein the therapeutic agent is either dissolved or suspended in the fatty base and/or the water soluble base.

6. A suppository according to claim 1, wherein the anti-cholinergic agent is at least one of darifenacin, fesoterodine, flavoxate, mirabegron, oxybutynin, probantheline, solifenacin, tolterodine, trospium, or a mixture of any thereof.

7. A suppository according to claim 1, wherein the mixed activity anti-cholinergic agent is at least one of amineptine, amitriptyline, amiltriptylinoxide, amoxapine, butriptyline, cidoxepin, clocapramine, clomacran, clomipramine, daledalin, demexiptiline, desmethylamitriptyline, desipramine, dibenzepin, dimetacrine, dotheipin, doxepin, fluacizine, N-methyl-doxepin, imipramine, iprindole, lofepramine, maprotiline, melitracin, metapramine, mirtazepne, nitroxazepine, norclolipramine, nortriptyline, noxiptyline, opipramol, perlapine, pizotyline, propizepine, protriptyline, quinupramine, reboxetine, tianeptine, trimipramine, or a pharmaceutically acceptable derivative or bioisostere of any thereof.

8. (canceled)

9. A suppository according to claim 1, wherein the fatty base is selected from the group consisting of paraffin, theobroma oil, modified theobroma oil, hydrogenated vegetable oils, cocoa butter, celluloses, polyvinyl alcohol, polyvinylpyrrolidone, polyacrylamide, polyphosphourethanes, polyoxyl stearate, ethylene oxide polymers, glyceryl monostearate, triglycerides, hexanone, and a mixture of any thereof.

10. A suppository according to claim 1, wherein the water soluble base is selected from the group consisting of gelatins, glycerinated gelatins, polyethylene glycols (PEGs), glycerol s, celluloses, polyvinyl alcohol, polyvinylpyrrolidone, polyacrylic acid, polyacrylamide, polyphosphourethanes, polyoxyl stearate, ethylene oxide polymers, starches, modified starches, and a mixture of any thereof.

11. A suppository according to claim 1, wherein the suppository additionally contains a suspending agent.

12. A suppository according to claim 1, wherein the therapeutic agent is included in a concentration range of about 0.1% to 10%.

13. (canceled)

14. (canceled)

15. A suppository according to claim 1, having a configuration selected from the group consisting of a baseball bat, a cylinder, a cone, a rocket, and an ellipsoid.

16. A suppository according to claim 1, wherein the suppository has a length of about 5_mm to about 50 mm, and a traverse dimension of about 1 mm to about 10 mm.

17. A suppository according to claim 1, wherein the suppository has a weight of about 10 mg to about 1000 mg.

18. A suppository according to claim 2, wherein the suppository is adapted to melt/dissolve over a period of 5-60 minutes once inserted into the urethra.

19. (canceled)

20. (canceled)

21. A suppository according to claim 1, wherein the anti-cholinergic agent is oxybutynin.

22. A suppository according to claim 1, wherein the fatty base comprises a hydrogenated vegetable oil.

23. A method for treating one or more symptoms of overactive bladder comprising administering to a person in need thereof the suppository of claim 1.

24. The method according to claim 21, wherein the one or more symptoms comprise nocturia.

25. A suppository according to claim 11, wherein the suspending agent is silica.

26. A suppository according to claim 25, wherein the suppository comprises silica at a concentration of about 0.1% to about 5%.
